(19) United States
(12) Patent Application Publication
(10) Pub. No.: US 2002/0128701 A1
(43) Pub. Date: Sep. 12, 2002

(54) Low profile expandable hoop support device for flexible tubes

(76) Inventor: R. Edward Winters, (US)

Correspondence Address: JOHN M. BRANDT, 60 THAXTER ST, HINGHAM, MA, 02043, US

(21) Appl. No.: 09/846,565

(22) Filed: Apr. 27, 2001

Related U.S. Application Data
(60) Provisional application No. 60/200,262, filed on Apr. 28, 2000.

(51) Int. Cl. A61F 2/06

(52) U.S. Cl. 623/1.11

(57) ABSTRACT

A hoop support device for insertion into flexible tubes or other body cavities designed to hold open these areas due to the hoop strength of the device while maintaining flexibility to bend with the area within which it is placed. The device, consisting of one or more hoops of coiled wire of any desirable material, either metallic or non metallic, is preferably non magnetic. The hoop or series of hoops is preformed to match the structure within which it is to be placed by utilizing a length of coiled material and forming it over a mandrel of desired shape and then instilling a memory in the resulting shape by heat treatment or other suitable means such that the formed shape will be reformed after placement into the desired configuration. For placement, the formed shape may be reformed into a linear coil configuration by means of inserting the linear coil into a tube or onto a rod. The linear coil is then inserted into the area to be supported and the end of the coil positioned at the desired location such that upon removal of the rod or ejection from the tube, the pre-formed secondary shape will reform or re-establish itself within the area to be supported. The invention is suitable for medical applications, particularly vascular applications, but may be utilized for almost any support purpose on a micro or macro scale.

### CROSS REFERENCE TO RELATED APPLICATIONS

This application is a formal application based on the disclosure of the Provisional Application by the same inventor and of the same title, Serial No. 60/200,262, filed Apr. 28, 2000.

### BACKGROUND OF THE INVENTION

1. Field of the Invention

The invention relates to stents introduced into body cavities such as vessels, ducts, or other openings to provide patency by maintaining a lumenal opening through the stent.

2. Description of the Prior Art

Stents have become increasingly popular in recent years for the purpose of holding open coronary arteries after procedures such as angioplasty to avoid re-closure due to a flap that may be formed during the angioplasty procedure from disruption of the vessel wall or due to the rebound of the dilated area after the dilatation procedure. Likewise, stents have been utilized to hold open grafts and other body cavities wherein the wall integrity and hoop strength is insufficient or has lessoned and support is required to maintain an opening. Stents for coronary arteries are generally of two basic types, expandable or self-expanding. In the former case, the stent is delivered on an expandable balloon to the desired location and then expanded into position by inflation of the balloon. Alternatively, in the latter case the stent is compressed to the surface of the catheter and held in place or restrained from expansion until positioned in the desired area and the tube or other means of suppressing expansion is removed allowing the stent to expand into position. Both of the aforementioned procedures follow an angioplasty procedure to open a narrowing of the vessel. A multitude of designs have been patented each with claims as to the desirability of the features of the stents in maintaining position or flexure within the desired location. Despite these variations in design, at least in the coronary vascular applications, restenosis still occurs in the area of the stents. In addition, the stents being relatively rigid during and following placement preclude long stents from being placed through tortuous vessels. Once placed these stents are relatively rigid structures with abrupt changes in the rigidity of the vessel at the ends of the stents possibly contributing to the growth of cells at the junction which contribute to the restenosis. Lastly, the stents by necessity of being placed while riding on the outside surface of the catheter have a finite diameter when compressed which is slightly larger than the catheter on which they are delivered.

In addition, even those stents which have been delivered from within a catheter have a fairly large profile thus requiring a separate specially designed catheter for stent delivery which is different from the dilatation catheter. Because the design of most stents is from cutting a pattern into a tube which is then expanded to support a vessel, there is a finite limitation of the ratio of the tube size when compressed to the diameter of the expanded stent such that the compressed stent is relatively large either when delivered from within or from the outside of a catheter. Despite a wide variation in the patent literature of patterns cut into the tubular stents with consideration for flexibility during and following placement, there is a physical limitation to how much material can be removed with the pattern cut into a tube and the amount of support provided resulting in only slight variations in features of tubular stents as to the amount of support and flexibility available and the length of the stent that may be placed in a tortuous vessel.

Accordingly, it would be desirable to have a stent which provides adequate hoop strength to support the area within which it is placed, and a very small profile for placement coupled with the flexibility to deliver longer stents that would traverse the tortuous vessels through which they must pass and to remain flexible when placed to avoid the abrupt transition at the ends of the stent. In addition, the ability to bend and flex with the area within the body wherein the device is placed whilst retaining hoop strength to hold open the structure is particularly important in coronary applications wherein the coronary arteries are constantly being compressed and bent as the cardiac muscle contracts and changes shape. This can create irritability of the vessel lining at the transition between the flexible vessel and the rigid stent.

The coronary arteries are on the surface of the heart under a membrane, the pericardial sac, and are compressed during every contraction. Therefore having hoop strength while also having longitudinal/axial flexibility to bend with the flexing cardiac muscle is one aim of the present invention. In addition, the present invention has an extremely low profile for placement due to the construction of the device from a primary and then secondarily formed helical spring coil thus allowing the device to be placed through rather than from the outer surface of a catheter. The lower profile for delivery would accommodate stenting smaller diameter vessels and the flexibility during placement would allow for longer stents to be placed and even to place stents around curved sections of vessels since the flexibility of the stent would allow for following the contour of the bend of the vessel while maintaining the lumen opening. The self expanding nature of the present invention would likewise reduce the size and complexity of the delivery device or catheter and the present invention could even be placed through the lumen of the dilatation catheter which could remain in position following dilatation for delivery of the stent, thus eliminating addition catheter insertions and reducing the time for placement which would make the procedure inherently safer. Because of the unique dual coil design of the present invention, retrieval and repositioning of the stent would be possible by merely returning the stent to the delivery device, either tube or rod prior to release. With current designs it is not possible with many of the current stent designs either expandable or self-expanding to remove them following placement. With the present invention it would also be possible to reposition the stent prior to placement or alternatively to easily snare and remove the stent following placement prior to tissue in-growth. The present invention, would allow for variations in both the primary and secondary coil spacing such that flow would not be disrupted into side branches when stenting across the side branch due to the porous nature provided by the coil spacing. The present invention accommodates a wide variation in diameters, lengths, hoop strength and choice of materials, as well as, diameter, tensile, magnetic qualities, and radio opacity utilizing mono-filar or multi-filar primary coils, and secondary coil variations in shape, diameter, taper or straight coils of varying lengths.

A plurality of stent devices exist in the prior art with each having a unique design intended to overcome some of the problems inherent with stents, but each with the intent of maintaining patency of an opening. For example, Palmaz, in U.S. Pat. No. 5,102,417, discloses an expandable intraluminal graft, and method and apparatus for implanting an expandable intraluminal graft by use of an angioplasty balloon associated with a catheter to dilate and expand the lumen of a vessel.

Roubin, et al., U.S. Pat. No. 5,827,321, discloses a stent with varying flexibility along it's length by use of a plurality of annular elements with each annular element including a plurality of struts and apices connected to form an annular configuration. Each annular element has a compressed state and an expanded state, and has a longitudinal dimension which is smaller in the expanded state than in the compressed state so that the plurality of connecting members connect the apices of adjacent annular elements. The connecting members have a plurality of alternating segments that function to compensate for the smaller longitudinal dimension of each annular element in the expanded state.

U.S. Pat. No. 6,217,608, Penn, et al., shows an expandable stent comprising a proximal end and a distal end in communication with one another and a tubular wall disposed between the proximal end and the distal end providing a balance of lateral flexibility of the unexpanded stent and radial rigidity of the expanded stent for facilitating placement through greater flexibility during placement.

Thorud, et al. in U.S. Pat. No. 6,019,779 discloses a multi-filar, open and closed coil, tubular medical stent that is introduced to a site in a body lumen and released to expand at the site to provide a passageway through the stent lumen.

Gianturco in U.S. Pat. No. 4,580,568 describes a percutaneous endovascular stent and method for insertion utilizing a compressed zigzag pattern of stainless steel wire which expands when ejected from inside the lumen of a catheter.

Additionally, U.S. Pat. No. 6,183,507, Lashinski, et al., teaches the use of a stent with a variable degree of support by means of materials of varying yield strength.

Finally, U.S. Pat. No. 6,146,403 St. Germain teaches the use of a stent having varying outward radial force along its length.

The present invention is a novel approach to overcoming some of the limitations of current stents by providing a self-deploying device which has flexibility both during delivery and after placement and may be removed or replaced without complex catheter systems in a very low profile device for stenting smaller diameter, as well as, larger diameter vessels without limitations of length and without sacrificing support.

### SUMMARY OF THE INVENTION

The present invention may be summarized as a hoop support device for insertion into flexible tubes or other body cavities designed to hold open these areas due to the hoop strength of the device while maintaining flexibility to bend with the area within which it is placed. The device consisting of one or more hoops of coiled wire of any desirable material may be either metallic or non metallic and preferably non magnetic. The hoop or series of hoops is preformed to match the structure within which the device is to be placed by utilizing a length of coiled material and forming it over a mandrel of desired shape and then instilling a memory in the resulting shape by heat treatment or other suitable means such that the formed shape will be reformed after placement into the desired configuration. For placement, the formed shape may be reformed into a linear coil configuration by means of inserting the linear coil into a tube or onto a rod. The linear coil is then inserted into the area to be supported and the end of the coil positioned at the desired location such that upon removal of the rod or ejection from the tube, the pre-formed secondary shape will reform or re-establish itself within the area to be supported. The invention is suitable for medical applications, particularly vascular applications, but may be utilized for almost any support purpose on a micro or macro scale.

These and other features and objects of the invention will become clearer from the following drawings and description of the preferred embodiment.

### DESCRIPTION OF THE DRAWINGS

FIG. 1 illustrates the preferred embodiment loaded linearly onto a rod or core with the distal segment partially advanced off of the rod and assuming the pre-formed shape imparted to the secondary coils which are being formed as the linear coil is advanced, or the rod retracted;

FIG. 2 is likewise an illustration of the preferred embodiment emanating from within a tube in which it has been linearly loaded for delivering the stent coils;

FIG. 3 is a cross sectional view of a vessel or cavity with a loosely formed secondary coil to support the walls;

FIG. 4 exhibits a more tightly formed secondary coil which has been placed inside an opening to provide radial support to the structure;

FIG. 5 illustrates in a cross sectional view the linear coil within a tube that has been placed within a cavity and partially pushed out of the tube to assume the pre-formed shape of the secondary coil in radially supporting the walls of the structure;

FIGS. 6, 7 8 and 9 are drawings depicting a vessel with an occlusion or narrowing in FIG. 6 which has been dilated as seen in FIG. 7 and then radially supported in FIGS. 8 and FIG. 9 by releasing the coil stent from within a tube as in FIG. 8 or from a core or rod as seen in FIG. 9 into the area which has been dilated and is to be supported by the coil stent;

FIG. 10 is a view of the coil stent after placement in a bend in a vessel;

FIG. 11 depicts the coil stent in an area of the vessel with a side branch with an open spaced secondary coil at the side branch; and

FIG. 12 illustrates the coil stent after placement in a bifurcated vessel with two diameters formed in the secondary coil to accommodate the vessel diameter on either side of the bifurcation and open spacing at the bifurcation.

### DESCRIPTION OF THE PREFERRED EMBODIMENT

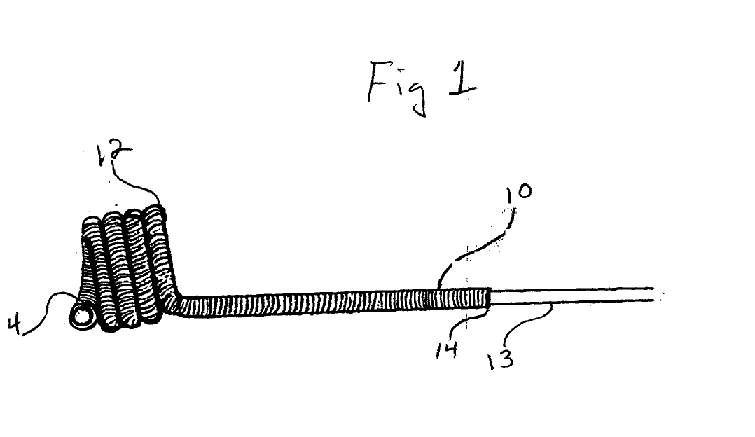

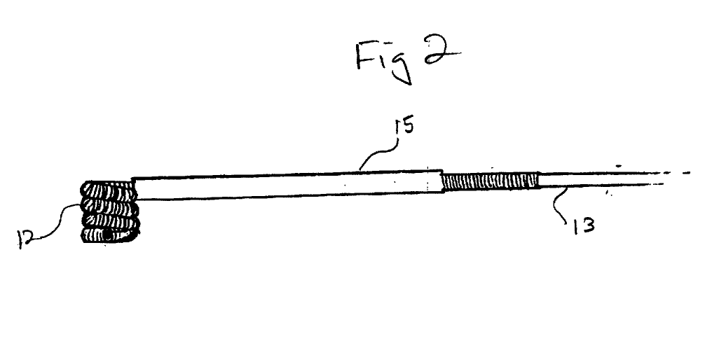

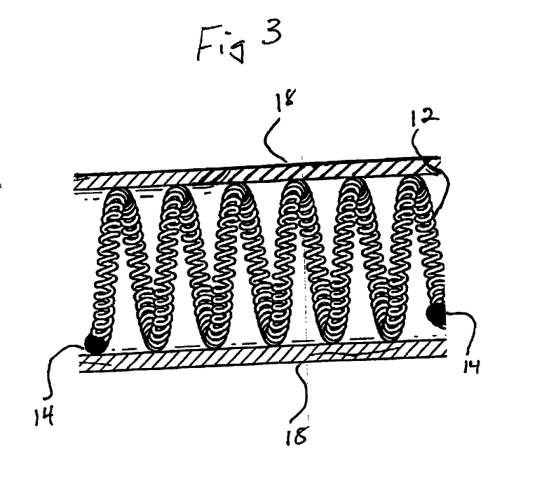

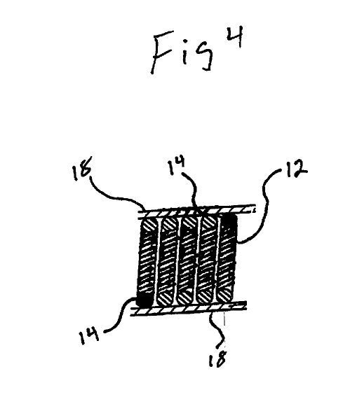

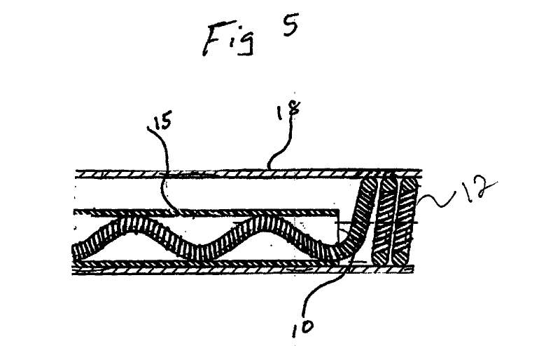

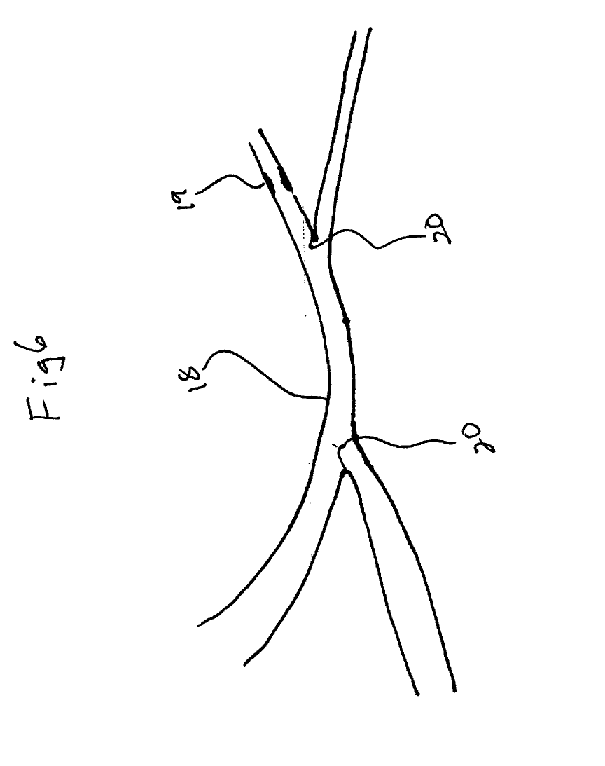

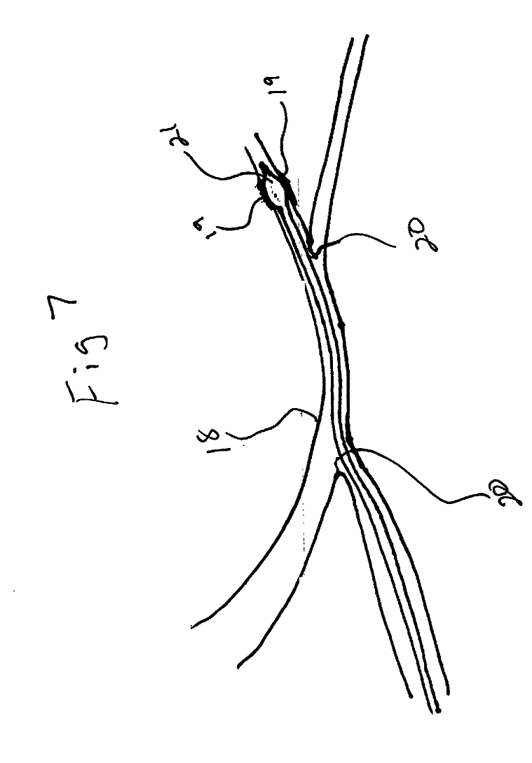

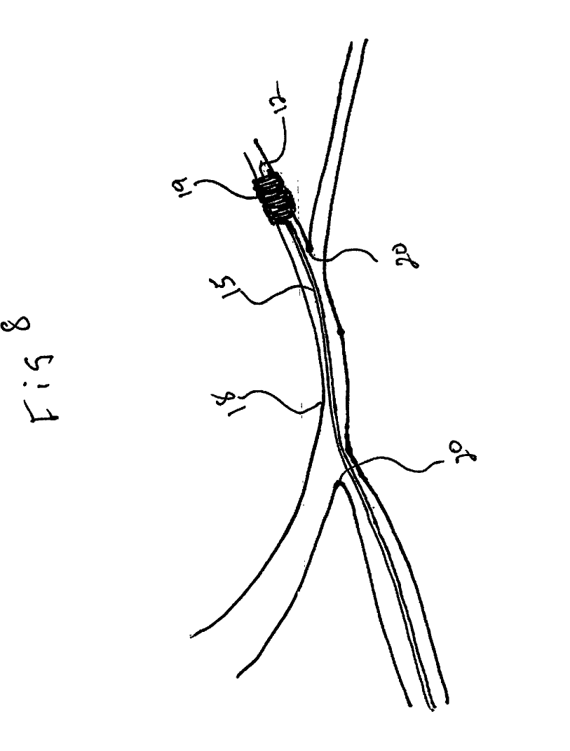

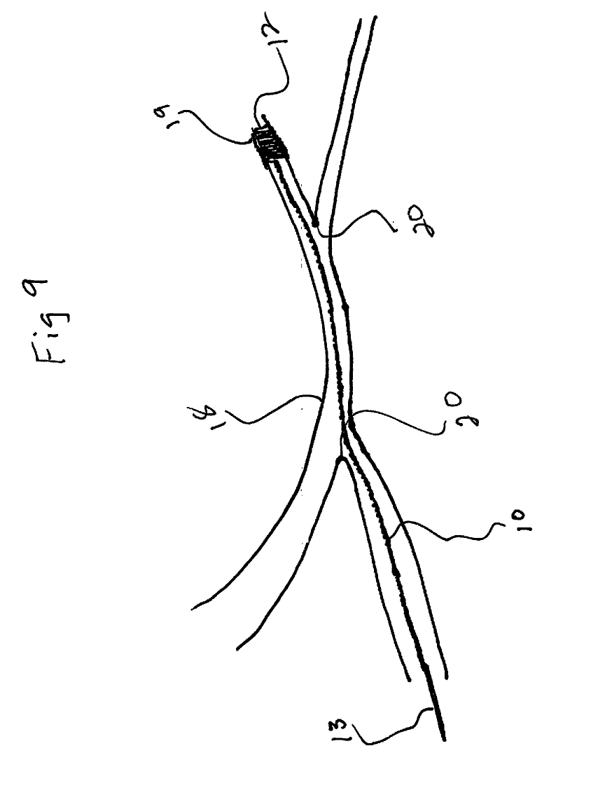

Referring to FIG. 1 there is shown an illustration of the preferred embodiment with the primary coil 10 loaded onto delivery core/rod 13 and the secondary coil 12 partially reformed to the predetermined and instilled shape as it is removed either by advancing the primary coils 10 or retracting the core/rod 13 from within the primary coils. The helically wound spring 10 may be a single or muti-filar coil of the same or dissimilar materials dependent upon the need for tensile strength and radio opacity. The spacing of the primary helical coils, as well as the secondary helical coils may be pre-set for the desired application, to provide increased support or greater porosity/coil spacing. In either instance, the profile of the primary coil 10 is quite small relative to the secondary coil 12 and the cavity to be supported. The ends of the primary coil may be rounded or formed into a ball 14 to reduce the likelihood of perforation or irritation of the vessel.

In FIG. 2 the secondary coil 12 is forming after being advanced from the delivery tube 15 and has been pre-loaded onto a core 13 prior to loading into the delivery tube/catheter 15, however it is not necessary to pre-load the primary coil onto a core prior to insertion into the delivery tube/catheter 15, since the tube/catheter 15 will accept the primary coil 10 in a linear configuration as it is advanced into the tube/catheter 15 and may be pushed from within the delivery tube 15 by a suitable rod which will fit within the delivery tube/catheter 15.

In FIG. 3 the loose coil configuration of the preformed secondary coil 12 is shown radially supporting a vessel 18 with ball ends 14 to protect the vessel lining. While in FIG. 4, the secondary coil 12 was pre-formed into a tight secondary coil configuration prior to placement in a vessel 18 and likewise has ball ends 14.

FIG. 5 illustrates in a cross sectional view of a vessel 18, the delivery tube 15 of small diameter relative to the size of the vessel 18 in which the secondary coil 12 is being released to re-form within the vessel 18. The wavy appearance of the primary coil 10 would be expected in a tube/catheter 15 with this much clearance but it is not necessary to have this much clearance within the delivery tube 15 and is shown for illustration of the linear appearance of the primary coil when loaded but after pre-forming the secondary coil 12 which has memory of the secondary shape that has been formed into the primary coil

FIGS. 6 through 13 illustrate a series of narrowings in vessels at various locations, wherein the coil stent may be placed by either a core/rod 13 or delivery tube/catheter 15 to support a lesion area 19 which has previously been narrowed and then dilated with an angioplasty balloon 21 prior to insertion of the secondary coil 12.

FIG. 6 illustrates a vessel 18 with two bifurcations 20 and a narrowing/lesion 19.

FIG. 7 illustrates the vessel 18 with two bifurcations 20 and a lesion 19 which has been dilated by angioplasty balloon 21.

FIG. 8. shows the vessel 18, delivery tube 15 and the formation of the secondary coils 12 to radially support the dilated lesion 19.

In FIG. 9 is illustrated the vessel 18 with the primary coil 10 loaded onto the rod/core 13 and secondary coil 12 formed in the area of the lesion 19 being pushed off of the delivery rod/core 13 to support the lesion 19.

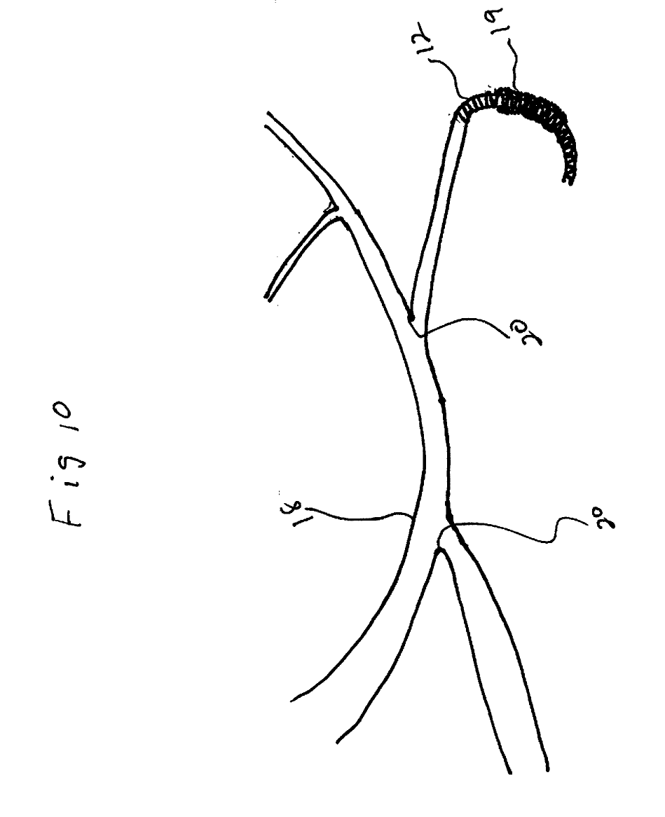

By using variations of the secondary coil pre-set shape and spacing, as well as, preset diameter it is possible to support a variety of lesions in either curved areas or at side branches and bifurcations as illustrated in FIGS. 10, 11, and 12.

Shown in FIG. 10 is a secondary coil 12 formed in a curved section of a vessel 18 at the site of a lesion 19.

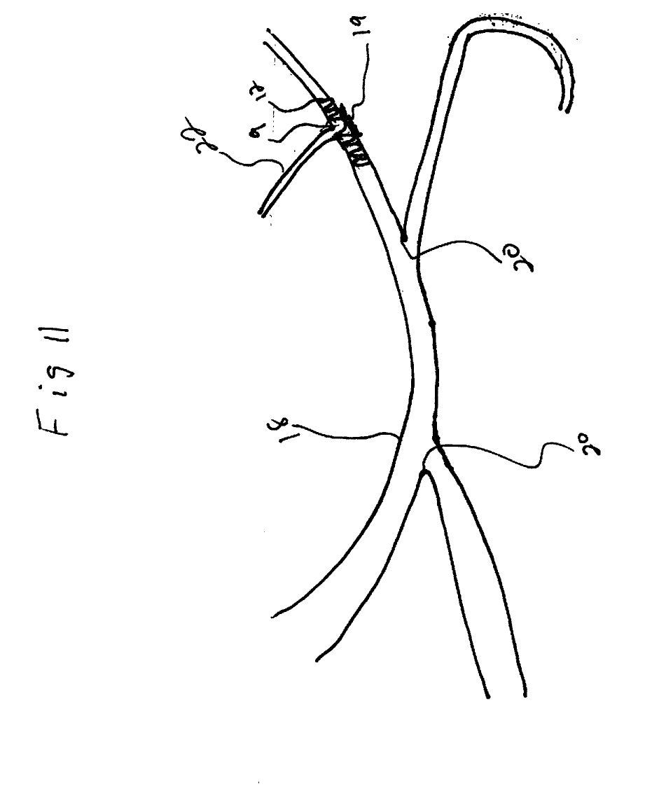

FIG. 11 likewise shows a secondary coil 12 with open spacing 9 formed in vessel 18 at the site of the lesion 19 at side branch 22 with open spacing 9 to allow for flow down the side branch 22.

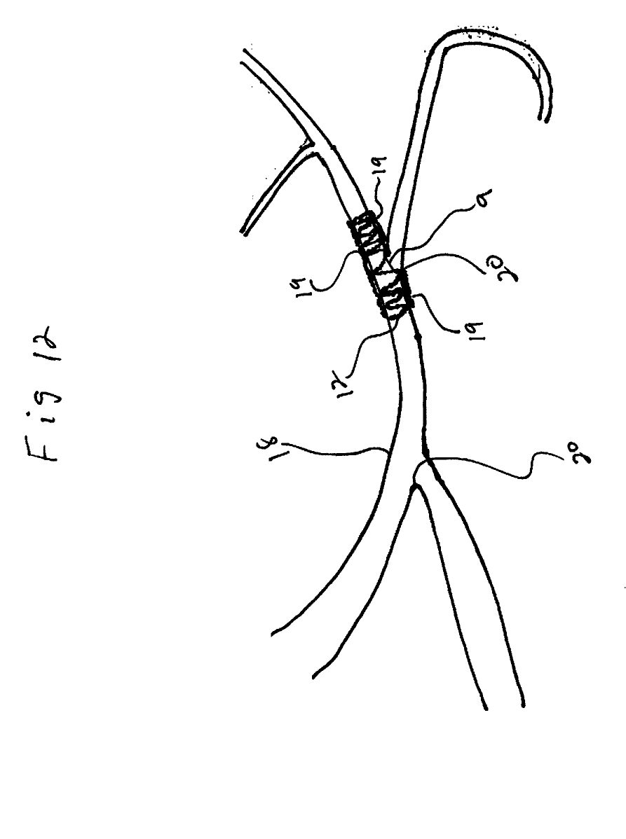

FIG. 12 illustrates the positioning of secondary coil 12 with open spacing 9 in a bifurcation 20 at the site of a lesion 19 in vessel 18.

As variations in the above description will now become obvious to those skilled in the art, the invention is accordingly, the invention is defined by the following claims.

What is claimed is:

1. An expandable hoop support for flexible tubing comprising in combination:
a. A preformed hoop composed of a coil of material having memory retaking properties; and
b. Cylindrical delivery means for constraining said coil into a linear configuration whereby when said delivery means and said coil are inserted into said tube and said delivery means is then removed, said hoop will then reconfigure from a constrained linear configuration to the original preformed hoop configuration.

2. The apparatus of claim 1 wherein said delivery means is a rod arranged to fit within said coil.

3. The apparatus of claim 1 wherein said delivery means is a tube arranged to fit over said coil.

4. The apparatus of claim 1 wherein said hoop comprises a stent.

5. The procedure for opening a coronary artery comprising in combination:
a. Providing a preformed hoop composed of a coil of material having memory retaining properties;
b. Providing cylindrical delivery means for constraining said coil into a linear configuration;
c. Inserting said hoop and said delivery means into said artery; and
d. Removing said delivery means whereby said hoop remains in said artery to support said artery in an open position.

6. The procedure of claim 5 wherein said delivery means is a rod arranged to fit within said coil.

7. The procedure of claim 5 wherein said delivery means is a tube arranged to fit over said coil.
